(19) United States
(12) Patent Application Publication
(10) Pub. No.: US 2008/0031873 A1
(43) Pub. Date: Feb. 7, 2008

(54) Humanized monoclonal antibody 31.1 as an anticancer agent

(76) Inventors: Jeffry Fasick, Bronx, NY (US); Nikos Panayotatos, Orangeburg, NY (US); Kwong Y. Tsang, Bethesda, MD (US)

Correspondence Address: BAKER BOTTS L.L.P., 30 ROCKEFELLER PLAZA, 44TH FLOOR, NEW YORK, NY, 10112-4498, US

(21) Appl. No.: 11/644,048

(22) Filed: Dec. 22, 2006

Related U.S. Application Data
(63) Continuation of application No. PCT/US05/23317, filed on Jun. 30, 2005.
(60) Provisional application No. 60/584,400, filed on Jun. 30, 2004.

(51) Int. Cl. A61K 39/395; A61P 35/00; C12N 5/06; C12P 21/08

(52) U.S. Cl. 424/133.1; 435/326; 435/69.6

(57) ABSTRACT

The present invention is directed to a CHO cell expression system for high level expression of a chimeric 31.1 monoclonal antibody specific for a human carcinoma-associated protein antigen. The present invention also provides a pharmaceutical composition comprising chimeric 31.1 monoclonal antibody derived from the CHO cells of the invention for use in immunotherapy or immunodiagnosis.

The present invention is based, at least in part, on the discovery that a humanized chimeric version of murine monoclonal antibody 31.1 has anti-tumor activity, in vitro and in vivo, against human pancreatic cancer cells. The invention is also based on the discovery of a CHO expression system for high level expression MAb 31. 1, which may be used to produce compositions of antibody which are free of serum contaminants.

### BACKGROUND OF THE INVENTION

Monoclonal antibody technology has made it possible to obtain pure antibody populations which permit purification and characterization of various tumor markers and tumor-associated antigens that are useful for immunodiagnostics or immunotherapy. A number of monoclonal antibodies have been described that have varying degrees of selectivity for tumor antigens versus normal cell surface markers. Some of these tumor antigens are broadly represented across several or many tumor types, whereas others appear to be limited to one type of tumor.

Tsang et al. have described one such monoclonal antibody, referred to as monoclonal antibody 31. 1, which recognizes an antigen which has been found to be associated with colon cancer and certain breast and ovarian epithelial tissues (benign and malignant) (“Monoclonal Antibodies to Human Colon Carcinoma Associated Antigens,” Intl. Symp. Biotech in Clin. Med., Rome, Italy, Apr. 13-15, 1987; Arlen et al., 1998, Crit. Rev Immunol, 18:133-8, U.S. Pat. No. 5,688,657 by Tsang and Arlen). U.S. Pat. No. 5,688,657 also reported that murine MAb 31.1 recognized PAN-I and MIA pancreatic cell lines, but failed to react with cells of the HS766T pancreatic cell line and did not react with either of two pancreatic carcinoma fresh tumor tissues.

To develop a reagent that is compatible for use in humans, a chimeric recombinant monoclonal antibody was constructed which incorporated the variable domains of both the heavy and light chains of the murine 31.1 onto the conserved domains of a human IgG molecule. Construction of such a chimeric 3 1 I antibody reduces the ability of a human patient to mount an immune response against foreign mouse monoclonal antibodies, e.g., “human anti-mouse antibodies” (HAMA). Chimerization of 31.1 was first described in Arlen et al. (1998, Crit. Rev Immunol, 18:133-8).

The importance of developing an antibody which is not immunogenic is underscored by the clinical experience with humanized antibody A33. The A33 MAb, originally murine monoclonal antibody AS 33, was raised against the pancreatic cell line ASPC-1, but subsequently developed for use in colorectal cancer (U.S. Pat. No. 5,160,723 by Welt et al., U.S. Pat. No. 5,643,500 by Welt et al., U.S. Pat. No. 6,346,249 by Barbas, III et al.). As shown by experimental examples set forth below, there is data consistent with monoclonal antibodies A(S)33 and 31.1 binding to the same antigen. However, as reported in Welt et al., 2003, Clinical Cancer Res. 9:1338-1346, in a Phase I study of humanized antibody A33, eight of eleven patients developed a human antihuman antibody (HAHA) response, so that the clinical trials were discontinued. Of note, three patients who remained HAHA negative achieved a radiographic partial response, with a reduction of serum carcinoembryonic antigen from 80 to 3 ng/ml, and of four patients with radiographic evidence of stable disease, two showed significant reductions (>25%) in serum carcinoembryonic antigen (Welt et al., 2003, Clinical Cancer Res. 9:1338-1346).

In addition to the continued need for non-immunogenic therapeutic antibodies, the successful development of monoclonal antibodies for use as immunodiagnostic or immunotherapeutic reagents relies on the ability to produce large quantities suitable for human administration. For the purpose of obtaining Federal Drug Administration (FDA) approval it is essential that monoclonal preparations have as few contaminants as possible. Chinese Hamster Ovary (CHO) cells have the advantage of providing both high level expression and the ability to grow under serum free conditions thereby reducing contamination of antibody preparations.

### SUMMARY OF INVENTION

The present invention relates to a CHO expression system designed for high level expression of humanized chimeric 31.1 monoclonal antibodies (hereafter, simply “chimeric”) for use as immunodiagnostic and/or immunotherapeutic reagents. The present invention further relates to a variant chimeric 31.1 monoclonal antibody discovered while generating the CHO expression system of the invention. In addition, the present invention provides for the use of chimeric 31.1 monoclonal antibodies as anticancer agents, particularly against pancreatic cancer.

The present invention is directed to a CHO cell expression system for high level expression of chimeric 31.1 monoclonal antibody (“Chi 31.1 MAb”). The CHO-produced Chi 31.1 MAb (“CHO Chi 31.1 MAb”) was found to recognize antigen expressed on the surface of colon and pancreatic carcinomas. CHO cells expressing a variant chimeric 31.1 have been deposited at ATCC and assigned ATCC Patent Deposit Designation PTA-5712. The above deposit was made at American Type Culture Collection, 12301 Parklawn Drive, Rockville, Md. 20862 USA on Dec. 30, 2003.

The present invention also provides a pharmaceutical composition comprising chimeric 31.1 monoclonal antibody derived from the CHO cells of the invention for use in immunotherapy or immunodiagnosis of human carcinomas. In such instances, the chimeric 31.1 monoclonal antibody may be conjugated to a reporter molecule, a cytotoxic radioisotope, a cytotoxic drug, or a cytotoxic protein, in a suitable excipient.

The present invention further provides for variant chimeric 31.1 monoclonal antibodies having amino acid substitutions at different positions in the protein.

The present invention also is directed to a method of targeting cytotoxicity to cells expressing a carcinoma-associated antigen, comprising:
(a) delivering to the cells a chimeric 31.1 monoclonal antibody derived from the CHO cells of the invention and a cytotoxic effector agent; and
(b) allowing the cytotoxicity to occur.

In a specific embodiment of the invention, the effector agent may be complement, or effector cells active in ADCC. Alternatively, antibodies conjugated with a cytotoxic radionuclide, drug or protein may be used directly.

The present invention also provides diagnostic methods for detecting expression of human carcinoma associated antigen within a subject. Detection of such expression indicates the presence of a carcinoma in said subject. For diagnostic purposes, the chimeric 31.1 monoclonal antibody derived from the CHO cells of the invention is covalently or non-covalently labeled with, i.e., labeled with, a reporter molecule. Such reporter molecules include but are not limited to fluorescent and bioluminescent molecules or radiolabeled molecules. The method of the invention comprises contacting the test subject with labeled chimeric 31.1 monoclonal antibody and further imaging or assaying to detect expression of the human carcinoma-associated protein antigen.

Cells expressing human carcinoma—associated protein antigen can be imaged using a number of methods well known to those of skill in the art. Such methods include, for example, use of a CCD low-light monitoring system, positron emission tomography (PET), single photon emission computed tomography (SPECT), magnetic resonance imaging (MRI), and endoscopic optical coherence tomography.

### BRIEF DESCRIPTION OF THE DRAWINGS

FIG. 1 is a depiction of the pDCM/dhfr+H+L (pIBS1) plasmid used to generate CHO cells expressing chimeric 31.1 monoclonal antibody.

FIG. 2A-F is the nucleic acid sequence of pIBS1.

FIG. 3A is the nucleotide sequence of Mab 31.1 Heavy Chain, wherein the Kozak sequence is underlined and variant nucleotides are indicated in boldface text, annotated to show the variation.

FIG. 3B is the amino acid sequence of MAb Chimeric 31.1 heavy chain with variant amino acids indicated in boldface text, annotated to show the variation.

FIG. 4A is the nucleotide sequence of MAb Chimeric 31.1 Light chain including underlined Kozak Sequence.

FIG. 4B is the amino acid sequence of Mab chimeric 31.1 light chain.

FIG. 5 depicts results of staining various colon and pancreatic adenocarcinoma tumor cell lines with CHO Chi 3 1.1 MAb and murine 31.1 MAb.

FIG. 6A-B depicts cytotoxic effects of CHO Chi 31. 1 MAb on (A) colon carcinoma cell line LS 174T and (B) pancreatic cell line AsPC1, where MAb UPC-10 is a murine IgG2a kappa antibody with specificity against beta 2,6 fructosan.

FIG. 7A-B depicts complement directed cytotoxicity of CHO 31.1 MAb against (A) colon carcinoma cell line LS 174T and (B) pancreatic cell line AsPC1.

FIG. 8 depicts the results of experiments in which the effect of CHO 31.1 MAb was tested for activity in inhibiting tumor growth of LS 174T colon carcinoma cells in vivo in a nude mouse tumor model.

FIG. 9 depicts the results of experiments in which the effect of CHO 31.1 MAb (two doses) was tested for activity in inhibiting tumor growth of AsPC-1 pancreatic carcinoma cells in vivo in a nude mouse tumor model.

FIG. 10 depicts the results of experiments in which the effect of CHO 31.1 MAb (three doses) was tested for activity in inhibiting tumor growth of AsPC-1 pancreatic carcinoma cells in vivo in a nude mouse tumor model.

FIG. 11 depicts the results of cell binding affinity studies.

FIG. 12 depicts the results of studies in which CHO cells were transiently transfected with human glycoprotein A33 cDNA, and then the binding of biotinylated CHO 31.1 and biotinylated mAb A33 were compared.

FIG. 13 depicts the results of studies in which CHO cells were transiently transfected with human glycoprotein A33 cDNA, and then the binding of CHO 31.1 and mAb A33 were compared by FACS analysis

FIG. 14 depicts the inhibition of AsPC-1 pancreatic cell colony formation in soft agar by CHO 31.1 MAb.

### DESCRIPTION OF THE PREFERRED EMBODIMENTS

The present invention provides a CHO expression system and antibodies derived from such a system that are capable of specific binding to human carcinoma-associated antigens expressed on the surface of carcinoma cells. Such antigens have also been found to be expressed on the surface of colon and pancreatic carcinomas. The expression of carcinoma-associated antigens on the surface of colon and pancreatic carcinoma, but not on the surface of their normal counterparts, provides a method for selected targeting of cytotoxicity to such cells. Thus, the antibodies of the invention can be used for therapeutic purposes in subjects having or developing colon or pancreatic carcinoma The present invention further provides chimeric Mab 31.1 antibodies, including variant chimeric Mab 31.1, which are derived from the CHO expression system of the invention. Because of the growth properties of CHO cells, the expression system of the invention provides a means for generating large quantities of chimeric 31.1 antibodies such as variant chimeric 31.1 antibodies which are post-translationally modified in a manner similar to those expressed from human cells. The present invention further provides for “derivatives” of the antibodies of the invention which contain additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to other macromolecules.

The chimeric 31.1 antibodies of the present invention are specific for carcinoma-associated antigens, and CHO Chi 31.1 MAb has been observed to bind to tumor antigens present on certain colon cancer and pancreatic cancer cells. In view of the binding properties of murine MAb 31.1 and its chimeric equivalent, as described in U.S. Pat. No. 5,688,657, antibody compositions of the present invention may be expected to bind to certain breast and ovarian epithelial tissues. The immunogenicity of these antigens is expressed chiefly as cell-mediated immunity, measurable either by assay of delayed cutaneous hypersensitivity in vivo (“skin tests”), or by various in vitro assays of specific lymphocyte reactivity, such as lymphocyte proliferation or lymphocyte migration inhibition assays. For general principles of immunogenicity and description of various assays of specific immunological reactivity, see: Roitt, I., Essential Immunology, 6th Ed., Blackwell Scientific Publications, Oxford (1988); Roitt, I. et al., Immunology, C. V. Mosby Co., St. Louis, Mo. (1985); Klein, J., Immunology, Blackwell Scientific Publications, Inc., Cambridge, Mass. (1990); Klein, J., Immunology: The Science of Self-Nonself Discrimination, John Wiley & Sons, New York, N.Y. (1982); and Paterson, P. Y., Textbook of Immunopathology, Grune and Stratton, New York, (1986).

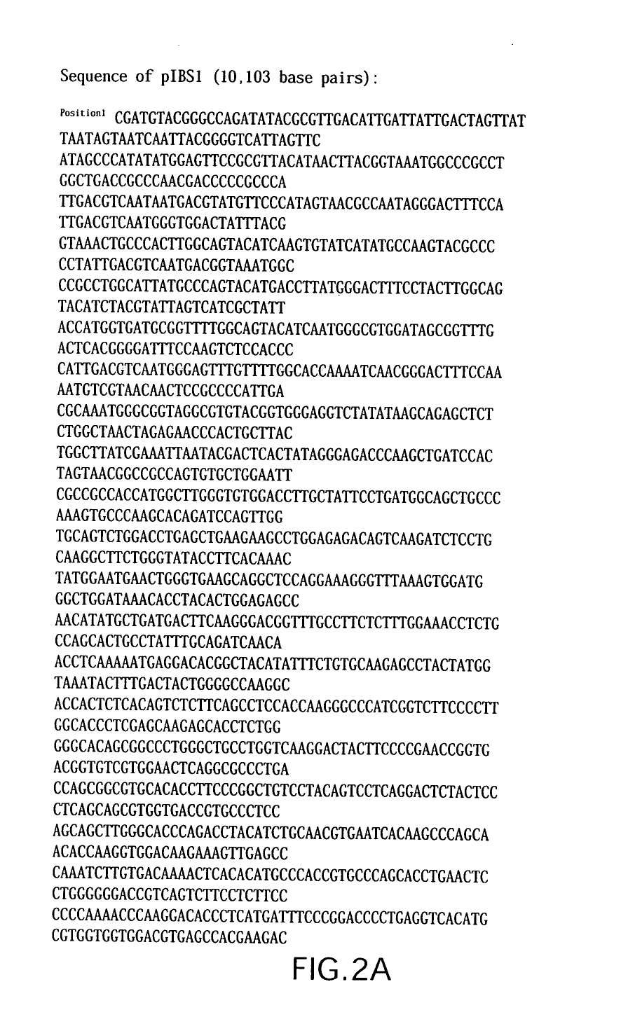

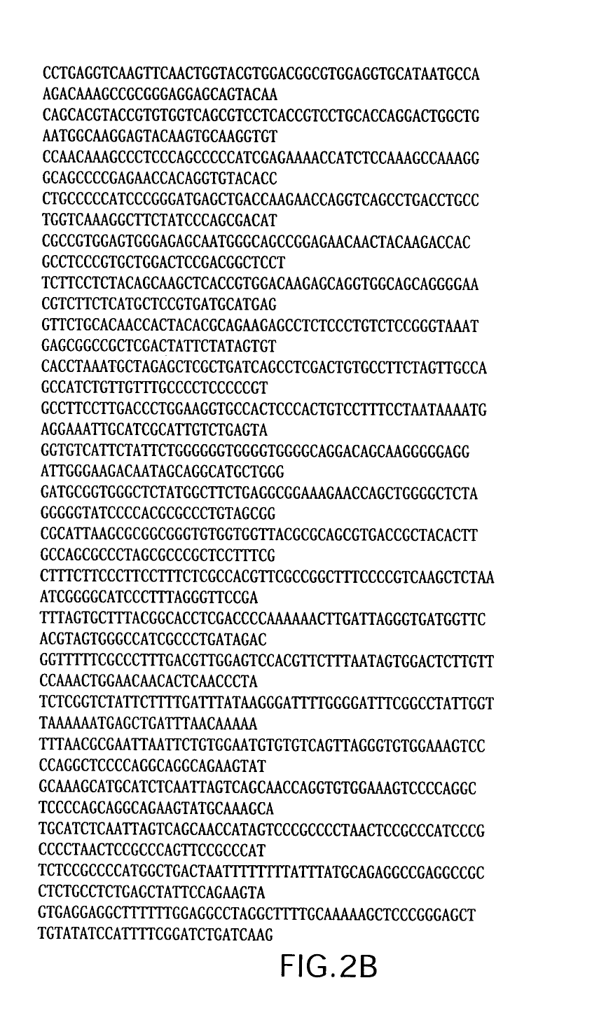

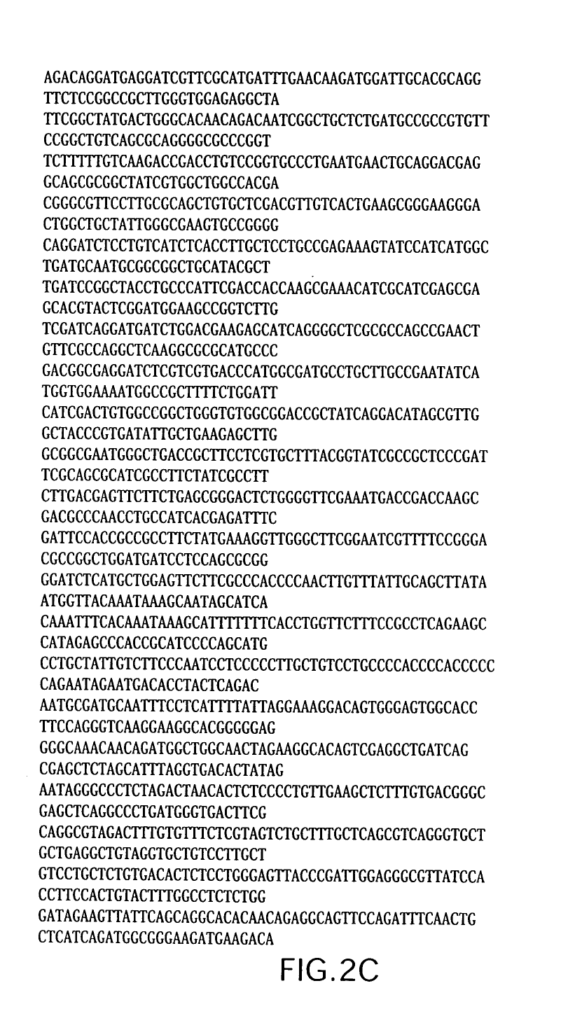

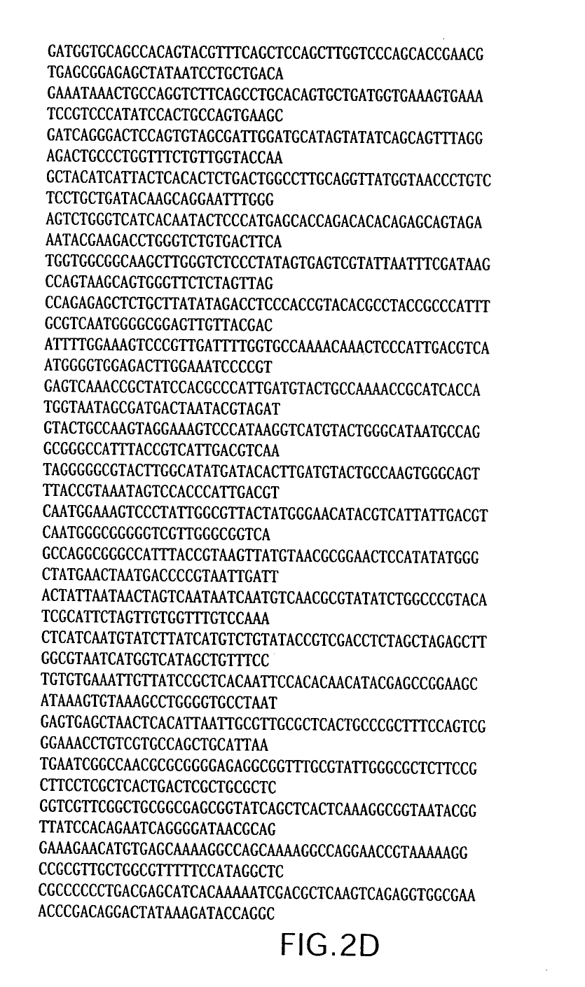

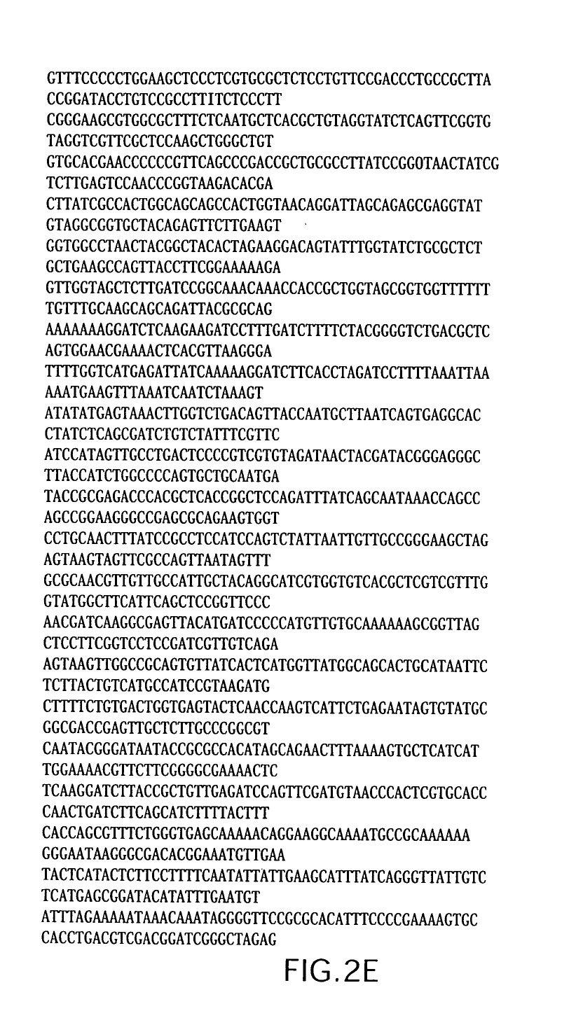

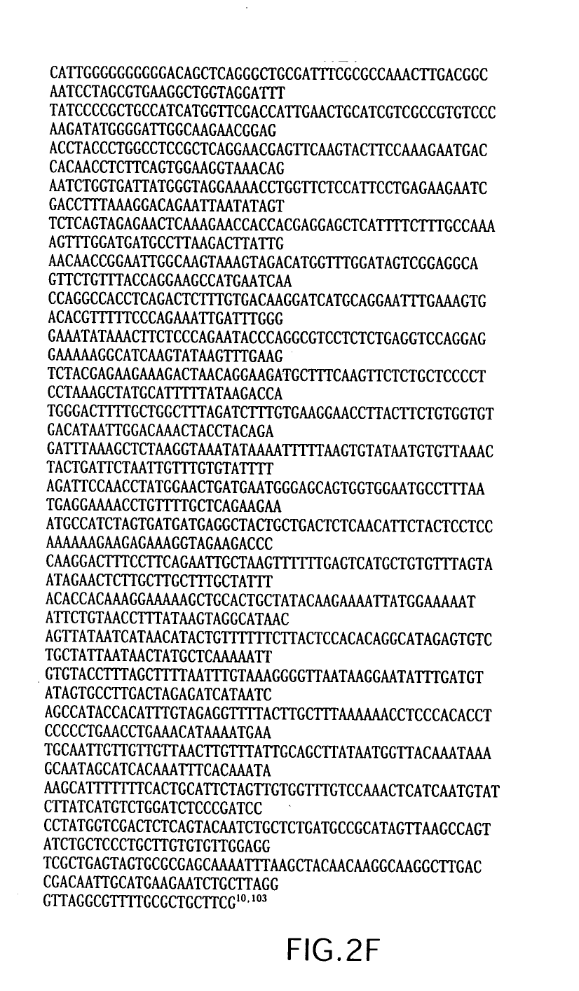

As used herein, the term “chimeric 31.1 monoclonal antibody” (Chi 31.1 MAb) includes monovalent, divalent or polyvalent immunoglobulins. The term Chi 31.1 MAb encompasses chimeric versions having the same amino acid sequence as Chi 31.1 disclosed in U.S. Pat. No. 5,688,657, MAbs comprising heavy and light chain variable regions having the same sequence as murine MAb 31.1 disclosed in U.S. Pat. No. 5,688,657, and also encompasses variants thereof In particular embodiments, the present invention provides for variant Chi 31.1 MAbs having sequences which are variants of the amino acid sequence of the chimeric 31.1 monoclonal antibody as produced by cells deposited with the American Type Culture Collection and assigned accession number CRL-12316. The present invention encompasses antibodies encoded by a nucleic acid having a sequence as set forth in FIG. 2A-F, as well as nucleic acids which are at least about 90 or at least about 95 percent homologous thereto, as determined using standard homology software such as BLAST or FASTA. In non-limiting embodiments, the present invention provides for a Chi 31.1 MAb encoded by a nucleic acid having a sequence as set forth in FIG. 2A-F except that the nucleic acid at position 428 is T or C; the nucleic acid at position 462 is T or C; the nucleic acid at position 473 is G or C; the nucleic acid at position 474 is A or T; the nucleic acid at position 475 is G or C; the nucleic acid at position 616 is T or C; the nucleic acid at position 839 is T or C; the nucleic acid at position 1049 is G or C; the nucleic acid at position 1261 is C or T; and/or the nucleic acid at position 1372 is T or C. In one specific, non-limiting embodiment, the present invention provides for a Chi 31.1 MAb encoded by a nucleic acid having a sequence as set forth in FIG. 2A-F except that the nucleic acid at position 428 is T ; the nucleic acid at position 462 is T; the nucleic acid at position 473 is G; the nucleic acid at position 474 is A; the nucleic acid at position 475 is G; the nucleic acid at position 616 is T; the nucleic acid at position 839 is T; the nucleic acid at position 1049 is G; the nucleic acid at position 1261 is C; and/or the nucleic acid at position 1372 is T.

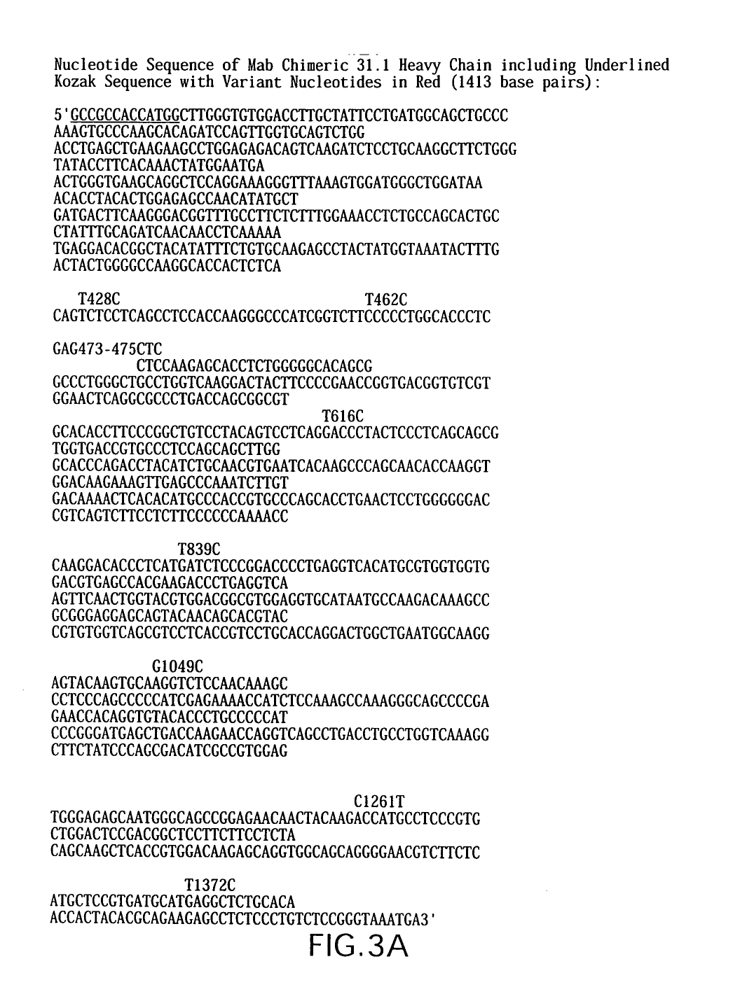

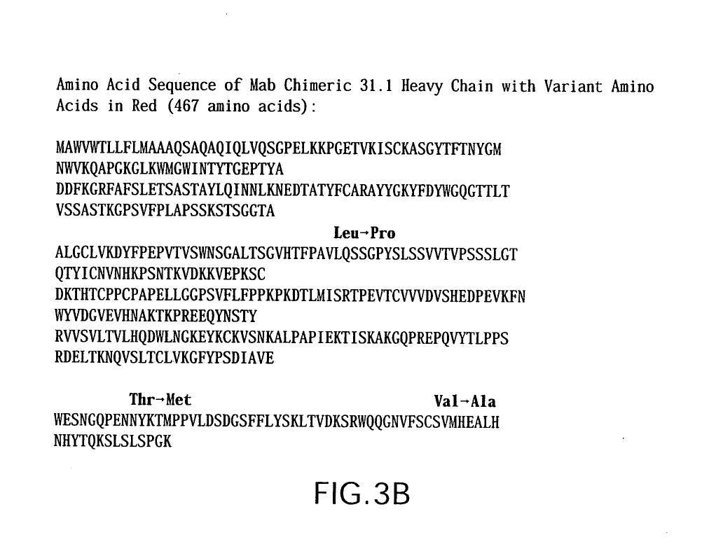

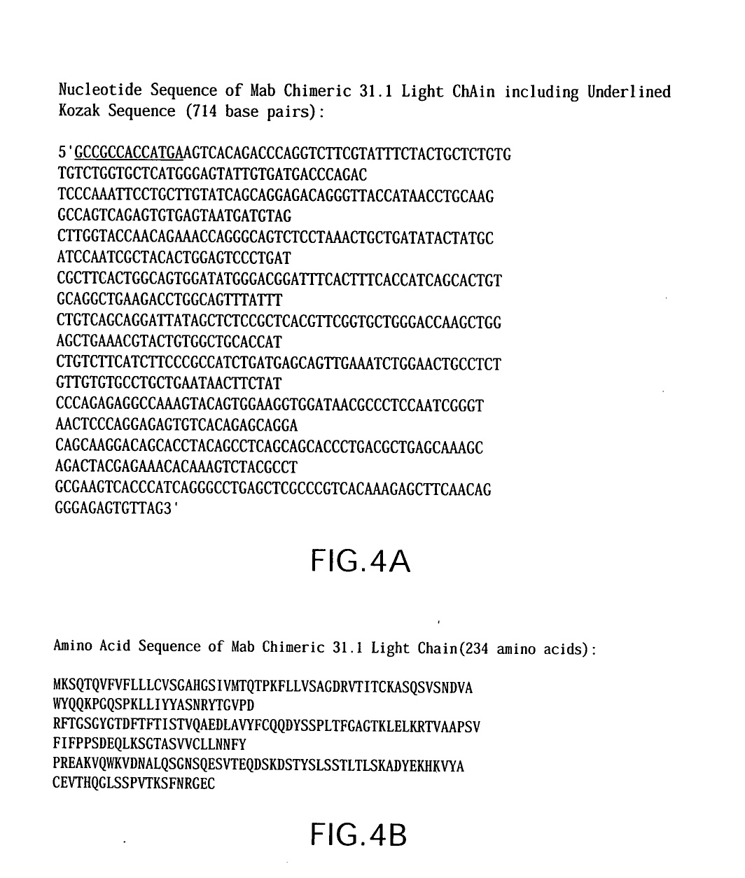

In particular, preferred embodiments, the present invention provides for variant Chi 31.1 MAb with heavy and light chains having amino acid sequences as set forth in FIGS. 3B and 4B. In one specific non-limiting embodiment, the variant chimeric 31.1 heavy chain nucleic acid sequence has the following mutations (31.1 nucleotide: position: substituted nucleotide):

T428C

T462C

G473C

A474T

G475C

T616C

T839C

G1049C

C1261T

T1372C

The nucleic acid substitutions of the variant chimeric 31.1 monoclonal antibody have been found to be neutral in their effect on the amino acid sequence of the predicted protein except for three such substitutions: T616C results in the amino acid substitution Leu→Pro; C1261T results in the amino acid substitution Thr→Met; and T1372C results in the amino acid substitution Val→Ala. The Val→Ala amino acid substitution is conservative in nature and most likely would have very minor effects, if any, on the mature protein. However, the Leu→Pro and Thr→Met amino acid substitutions are non-conservative by nature. The present invention encompasses variant Chi 31.1 MAbs having any one or more of these substitutions.

Variant chimeric 31.1 monoclonal antibody shows the same apparent affinity for antigen as those monoclonal antibodies produced from the previously described chimeric 31.1 sequences expressed in SP2/0 AG-14 cells (Arlen et al., 1998, Crit. Rev. Immunol. 18:133-8). Furthermore, all functional assays (i.e.cell flow cytometry, ADCC, ELISA, immunohistochemistry, western analysis) suggest that both the antigen binding domains as well as the constant regions of the variant chimeric 31.1 monoclonal antibody are able to form a functional IgG1 antibody. In mouse animal studies, the variant chimeric 31.1 monoclonal antibodies perform as well as the non-substituted chimeric 31.1 antibodies.

The present invention further provides a CHO expression system comprising CHO cells transfected with recombinant expression vehicles capable of expressing the heavy and light chains of the chimeric 31.1, or variant chimeric 31.1, monoclonal antibody. Such cells are designed for growth in conditioned media lacking serum components, such as fetal bovine serum, and high level expression of chimeric 31. 1 monoclonal antibodies and/or variant chimeric 31.1 monoclonal antibodies.

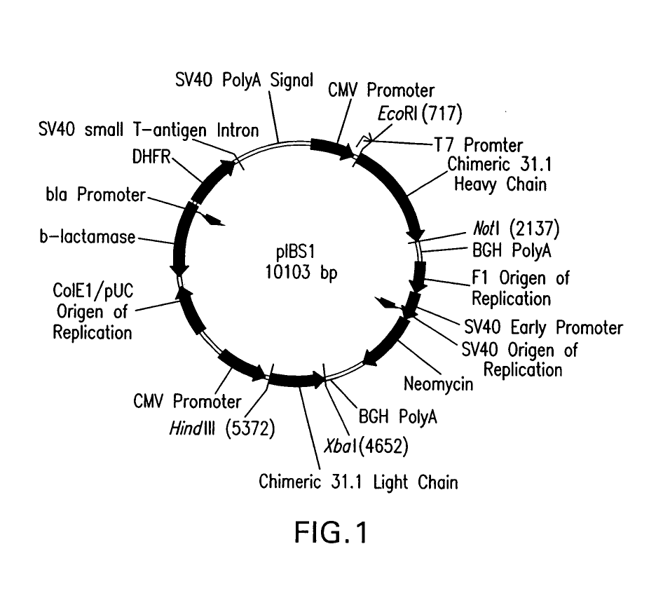

Expression vehicles to be utilized include plasmids or other vectors utilized in conjunction with CHO cells for expression of chimeric, or variant chimeric 31.1 monoclonal antibody. Preferred among these are vehicles carrying a functionally complete nucleic acid molecule capable of encoding the chimeric 31.1 monoclonal antibody heavy and light chains. Nucleic acid molecules capable of encoding the 31.1 heavy and light chains may be cloned into a single expression vector, i.e., pIBS 1 (FIG. 1), or alternatively, the heavy and light chain encoding nucleic acid molecules may be cloned into separate expression vectors. Many vector systems are available for the expression of cloned H and L chain genes in mammalian CHO cells (see Glover, D. M., ed., DNA Cloning, Vol. II, pp. 143-238, IRL Press, 1985 and U.S. patent No. which is incorporated herein in its entirety).

Gene expression elements useful for the expression of such nucleic acids include: (a)viral transcription promoters and their enhancer elements, such as the SV40 early promoter (Okayama, H. et al., Mol. Cell. Biol. 3:280 (1983)), Rous sarcoma virus LTR (Gorman, C. et al., Proc. Natl. Acad. Sci., USA 79:6777 (1982)), and Moloney murine leukemia virus LTR (Grosschedl, R. et al., Cell 41:885 (1985)); (b) splice regions and polyadenylation sites such as those derived from the SV40 late region (Okayama et al., supra); and (c) polyadenylation sites such as in SV40 (Okayama et al., supra).

Immunoglobulin heavy and light chain genes may be expressed as described by Weidle et al., Gene 51:21 (1987), using as expression elements the SV40 early promoter and its enhancer, the mouse immunoglobulin H chain promoter enhancers, SV40 late region mRNA splicing, rabbit β-globin intervening sequence, immunoglobulin and rabbit β-globin polyadenylation sites, and SV40 polyadenylation elements. For immunoglobulin genes comprised of part cDNA, part genomic DNA (Whittle et al., Protein Engineering 1:499 (1987)), the transcriptional promoter may be human cytomegalovirus (CMV), the promoter enhancers derived from CMV and mouse/human immunoglobulin, and mRNA splicing and polyadenylation regions derived from the native chromosomal immunoglobulin sequences.

Each fused gene is assembled in, or inserted into, one or more expression vectors. Recipient CHO cells capable of expressing the chimeric 31.1 immunoglobulin chain gene products are then transfected singly with a chimeric H or chimeric L chain-encoding gene, or are co-transfected with a chimeric H and a chimeric L chain gene. The transfected recipient cells are cultured under conditions that permit expression of the incorporated genes and the expressed immmunoglobulin chains or intact antibodies or fragments are recovered from the culture. In one embodiment, the genes encoding the chimeric H and L chains, or portions thereof, are assembled in separate expression vectors that are then used to co-transfect a recipient CHO cell. Alternatively, the genes encoding the chimeric H and L chains may be assembled in a single expression vector.

The expression vector carrying chimeric antibody constructs may be introduced into a CHO host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment (Johnston et al., Science 240:1538 (1988)).

The chimeric 31.1 monoclonal antibodies of the present invention, including their antigen-binding fragments and derivatives, have a multitude of uses relating to the therapy of colon and pancreatic cancer. Such uses are summarized in Schlom, J., Canc. Res., 46:3225-3238 (1986), which is hereby incorporated by reference.

A summary of the ways in which the chimeric 31.1 monoclonal antibodies of the present invention may be used therapeutically includes direct cytotoxicity by the antibody, either mediated by complement (CDC) or by effector cells (ADCC), or by conjugation to anti-tumor drugs, toxins, radionuclides. Additionally, the antibodies can be used for ex vivo removal of tumor cells from the circulation or from bone marrow.

The chimeric 31.1 monoclonal antibodies of the present invention, will also have uses relating to diagnosis of colon and pancreatic cancer. For diagnostic purposes, the chimeric 31. I monoclonal antibodies may be conjugated to a reporter molecule such as a fluorescent or bioluminescent molecule or radioactive label. Once the labeled chimeric 31.1 monoclonal antibody has been contacted with the test subject, cells can be imaged or assayed to detect expression of the human carcinoma—associated protein antigen thereby diagnosing the presence of a carcinoma within a host.

Cells expressing the human carcinoma-associated protein antigen can be imaged using a number of methods well known to those of skill in the art. Such methods include, for example, use of a CCD low-light monitoring system, positron emission tomography (PET), single photon emission computed tomography (SPECT), magnetic resonance imaging (MRI), and endoscopic optical coherence tomography. Some of these approaches are described in more detail below. Armed with the teachings provided herein, one of ordinary skill in the art will know how to use the chimeric 31.1 monoclonal antibodies of the present invention for diagnostic, monitoring or therapeutic purposes without undue experimentation.

The preferred animal subject of the present invention is a mammal. By the term “mammal” is meant an individual belonging to the class Mammalia. The invention is particularly useful in the treatment of human subjects.

By the term “treating” is intended the administering to subjects of the antibodies of the present invention or a fragment or derivative thereof for purposes which may include prevention, amelioration, or cure of colon or pancreatic cancer. “Amelioration” is defined herein to constitute one or more of the following:. stabilization of tumor size; slowing of tumor growth rate; decrease in tumor size or spread; reduction of pain or requirement for pain medication; slowing in rate of weight loss; decrease in carcinoembryonic antigen; or prolongation of pre-treatment expected survival.

The present invention provides for a method of treating pancreatic cancer, comprising administering, to a subject in need of such treatment, an effective amount of a humanized 31.1 monoclonal antibody. Humanized antibodies include chimeric antibodies as described herein as well as equivalent antibodies synthetically developed to be equivalent to human-derived or chimeric-derived sequences.

The pharmaceutical compositions of the present invention may be administered by any means that achieve their intended purpose. Amounts and regimens for the administration of chimeric 31.1 monoclonal antibodies, their fragments or derivatives can be determined readily by those with ordinary skill in the clinical art of treating colon or pancreatic cancer and related disease.

For example, administration may be by parenteral, subcutaneous, intravenous, intramuscular, intraperitoneal, transdermal, intrathecal, by buccal routes, or by local injection into a tumor or surgical site. Alternatively, or concurrently, administration may be by the oral route. The dosage administered will be dependent upon the age, health, and weight of the recipient, kind of concurrent treatment, if any, frequency of treatment, and the nature of the effect desired.

Compositions within the scope of this invention include all compositions wherein the chimeric 31.1 monoclonal antibody, fragment or derivative is contained in an amount effective to achieve its intended purpose. In particularly preferred, non-limiting embodiments, the present invention provides for compositions comprising CHO Chi 31.1 (and variant Chi3 1.1) which are free of serum-derived contaminants. For example, the present invention provides for compositions comprising CHO Chi 31.1 produced by genetically engineered CHO cells as deposited with the American Type Culture Collection and assigned accession number PTA-5712, where such compositions are free of serum-derived contaminants. While individual needs vary, determination of optimal ranges of effective amounts of each component is within the skill of the art. The effective dose is a function of the individual chimeric 31.1 monoclonal antibody, the presence and nature of a conjugated therapeutic agent, the patient and his clinical status, and can vary from about 10 ng/kg body weight to 10-100 mg/kg body weight. The preferred dosages comprise 0.1 to 10 mg/kg body weight.

In addition to the pharmacologically active compounds, the new pharmaceutical compositions may contain suitable pharmaceutically acceptable carriers comprising excipients and auxiliaries which facilitate processing of the active compounds into preparations which can be used pharmaceutically. Preferably, the preparations, contain from about 0.01 to 99 percent, preferably from about 20 to 75 percent of active compound(s), together with the excipient.

Preparations of the chimeric 31. I antibody, fragment or derivative of the present invention for parenteral administration, include sterile aqueous or non-aqueous solutions, suspensions, and emulsions. Examples of non-aqueous solvents are propyleneglycol, polyethyleneglycol, vegetable oil such as olive oil, and injectable organic esters such as ethyloleate. Aqueous carriers include water, alcoholic/aqueous solutions, emulsions or suspensions, including saline and buffered media, parenteral vehicles including sodium chloride solution, Ringer's dextrose, dextrose and sodium chloride, lactated Ringer's, or fixed oils. Intravenous vehicles include fluid and nutrient replenishers, such as those based on Ringer's dextrose, and the like. Preservatives and other additives may also be present, such as, for example, antimicrobials, anti-oxidants, chelating agents, and inert gases and the like. See, generally, Remington's Pharmaceutical Science, 16th ed., Mack Publishing Co., Easton, Pa., 1980.

In particular, the chimeric 31.1 antibodies, fragments and derivatives of the present invention are useful for treating a subject having or developing colon or pancreatic adenocarcinoma. Such treatment comprises administering,preferably parenterally, a single or multiple doses of the antibody, fragment or derivative, or a conjugate thereof. Such treatment may be performed concurrently or sequentially with other treatment regimens, including but not limited to surgical therapy, chemotherapy, and/or radiation therapy.

The chimeric 31.1 antibodies of the invention can be adapted for therapeutic efficacy by virtue of their ability to mediate ADCC and/or CDC against cells having CCAA associated with their surface. For these activities, either an endogenous source or an exogenous source of effector cells (for ADCC) or complement components (for CDC) can be utilized.

The chimeric 31.1 monoclonal antibodies of this invention, their fragments, and derivatives can be used therapeutically as immunoconjugates (see for review: Dillman, R. O., Ann. Int. Med. 111:592-603 (1989)). They can be coupled to cytotoxic proteins, including, but not limited to, Ricin-A, Pseudomonas toxin, Diphtheria toxin, and tumor necrosis factor. Toxins conjugated to antibodies or other ligands are known in the art (see, for example, Olsnes, S. et al., Immunol. Today 10:291-295 (1989)). Plant and bacterial toxins typically kill cells by disrupting the protein synthetic machinery.

The chimeric 31. I monoclonal antibodies of this invention can be conjugated to additional types of therapeutic moieties including, but not limited to, diagnostic radionuclides and cytotoxic agents such as cytotoxic radioisotopes, drugs and proteins. Examples of radionuclides which can be coupled to antibodies and delivered in vivo to sites of antigen include ²¹²Bi, ¹³¹I, ¹⁸⁶R, and ⁹⁰Y, which list is not intended to be exhaustive. The radioisotopes exert their cytotoxic effect by locally irradiating the cells, leading to various intracellular lesions, as is known in the art of radiotherapy.

Cytotoxic drugs which can be conjugated to chimeric 31.1 monoclonal antibodies and subsequently used for in vivo therapy include, but are not limited to, daunorubicin, doxorubicin, methotrexate, and Mitomycin C. Cytotoxic drugs interfere with critical cellular processes including DNA, RNA, and protein synthesis. For a fuller exposition of these classes of drugs which are known in the art, and their mechanisms of action, see Goodman, A. G., et al., Goodman and Gilman's The Pharmacological Basis of Therapeutics, 7th Ed., Macmillan Publishing Co., 1985. The chimeric 31.1 monoclonal antibodies of this invention may be advantageously utilized in coordination with other monoclonal or chimeric antibodies, or with lymphokines or hemopoietic growth factors, etc., which serve to increase the number or activity of effector cells which interact with the antibodies.

The chimeric 31.1 monoclonal antibodies, fragments, or derivatives of this invention, attached to a solid support, can be used to remove soluble carcinoma-associated antigens from fluids or tissue or cell extracts. In a preferred embodiment, they are used to remove soluble tumor antigens from blood or blood plasma products. In another preferred embodiment, the antibodies are advantageously used in extracorporeal immunoadsorbent devices, which are known in the art (see, for example, Seminars in Hematology, Vol. 26 (2 Suppl. 1) (1989)). Patient blood or other body fluid is exposed to the attached antibody, resulting in partial or complete removal of circulating carcinoma-associated antigens (free or in immune complexes), of carcinoma-associated antigens-bearing cells, following which the fluid is returned to the body. This immunoadsorption can be implemented in a continuous flow arrangement, with or without interposing a cell centrifugation step. See, for example, Terman, D. S. et al., J. Immunol. 117:1971-1975 (1976).

The chimeric 31.1 monoclonal antibodies of the present invention are also useful for immunoassays that detect or quantitate carcinoma-associated antigens or cells bearing carcinoma-associated antigens in a sample. Such an immunoassay typically comprises incubating a biological sample in the presence of a detectably labeled antibody of the present invention capable of identifying the tumor antigen, and detecting the labeled antibody that is bound in a sample.

In a specific embodiment of the invention, the chimeric 31.1 monoclonal antibodies may be used for in vivo imaging of colon, breast, and ovarian cancer using different reporter molecules and methods of labeling known to those of ordinary skill in the art. Examples of the types of reporter molecules that can be used in the present invention include radioactive isotopes, bioluminescent and chemiluminescent molecules, paramagnetic isotopes, and compounds which can be imaged by positron emission tomography (PET). CCD low-light monitoring system, single photon emission computed tomagraphy (SPECT) and magnetic resonance imaging (MRI). Those of ordinary skill in the art will know of other suitable labels for binding to the antibodies used in the invention, or will be able to ascertain such, using routine experiments. Furthermore, the binding of these labels to the antibody can be done using standard techniques common to those of ordinary skill in the art. For in vivo diagnosis, radionuclides may be bound to the antibody either directly or indirectly by using an intermediary functional group. Intermediary functional groups which are often used to bind radioisotopes which exist as metallic ions to the antibodies are the chelating agents, diethylene triamine pentaacetic acid (DTFA) and ethylene diamine tetraacetic acid (EDTA). Examples of metallic ions which can be bound to the antibodies of the present invention are ⁹⁹Tc, ¹²³I, ¹¹¹In, ¹³¹I, ⁹⁷ Ru, ⁶⁷ Cu, ⁶⁷Ga, ¹²⁵I, ⁶⁸Ga, ⁷²As, ⁸⁹Zr, and ²⁰¹Tl.

### EXAMPLE 1

### CHO Cells Expressing Chi Monoclonal Antibodies

To generate a high-level chimeric mAb 31.1 expressing CHO/dhfr-cell line, the chimeric 31.1 heavy and light chains were subcloned into the pDCM/dhfr expression vector. The resulting pDCM/dhfr+H+L (pIBS1) plasmid (the sequence of which is in FIG. 2A-F) was transfected into CHO/dhfr-cells (ATCC catalogue number CRL-9096) using a lipo-reagent. Stable transfected cells were selected with geneticin (G418) as well as HT-media. Clonal selection of high-level expressing cells was done in 96-well plates in the presence of G418 and methotrexate (MTX), and assayed by ELISA. After 2 months, twenty-five of the highest expressing clones were expanded into T-25 flasks. Amplification of the genes incorporating the pDCM/dhfr+H+L sequences was done by increasing the concentration of methotrexate added to the media After six months of selection and amplification, five high-level expressing clones were chosen for adaptation to suspension. After one month, two clones that adapted readily to suspension were chosen for adaptation to serum-free media. One such resulting clone is 3G9 which is deposited at American Type Culture Collection (ATCC), 12301 Parklawn Drive, Rockville, Md. 20862 USA and assigned ATCC Patent Deposit Designation PTA-5712.

The pIBS1 plasmid nucleic acid sequence includes alterations by which pseudo-Kozak sequences were introduced prior to the initiation codon of the heavy and light chains. In addition, the encoded heavy chain contains several amino acids which differ from those occurring in the original murine 31.1 antibody disclosed in U.S. Pat. No. 5,688,657. The mutations are depicted as the variant chimeric 31.1 amino acid sequences depicted in FIG. 3B.

All of the point mutations have been found to be neutral in their effect on the amino acid sequence of the predicted protein except for three: T616C results in the amino acid substitution Leu→Pro; C1261T results in the amino acid substitution Thr→Met; and T1372C results in the amino acid substitution Val→Ala. This last amino acid substitution is conservative in nature and most likely would have very minor effects, if any, on the mature protein. However, the first two amino acid substitutions are non-conservative by nature.

Variant chimeric 31.1 monoclonal antibodies show the same affinity for antigen as those monoclonal antibodies produced from the wild type chimeric 31.1 sequences expressed in SP2/0 AG-14 cells. Furthermore, all functional assays, i.e. cell flow cytometry, ADCC, ELISA, immunohistochemistry, western analysis, suggest that both the antigen binding domains as well as the constant regions are functional and able to form a functional IgG1 antibody. For example, in mouse animal studies, the CHO expressed variant chimeric 31.1 monoclonal antibodies, containing the amino acid substitutions, performed as well as the normal chimeric 31.1 antibodies generated from SP2/0 AG-14 cells.

### EXAMPLE 2

### CHO 31.1 MAb Binds to and Inhibits the Growth of Colon and Pancreatic Cancer Cells

Staining of normal cells and tissues. Immunohistochemistry of normal tissues and organs was done on both paraffin block as well as fresh frozen tissues. No observable staining was seen in most of the tissues examined. Exceptions were thyroid, jejunum, stomach, liver, salivary gland, parotid, colon, skin, adrenal, and thymus where minimal staining was observed. The intensity of antibody staining (0, no staining, +3, strong staining) as well as the tissue membrane structure that stained with antibody is shown in Table 1.

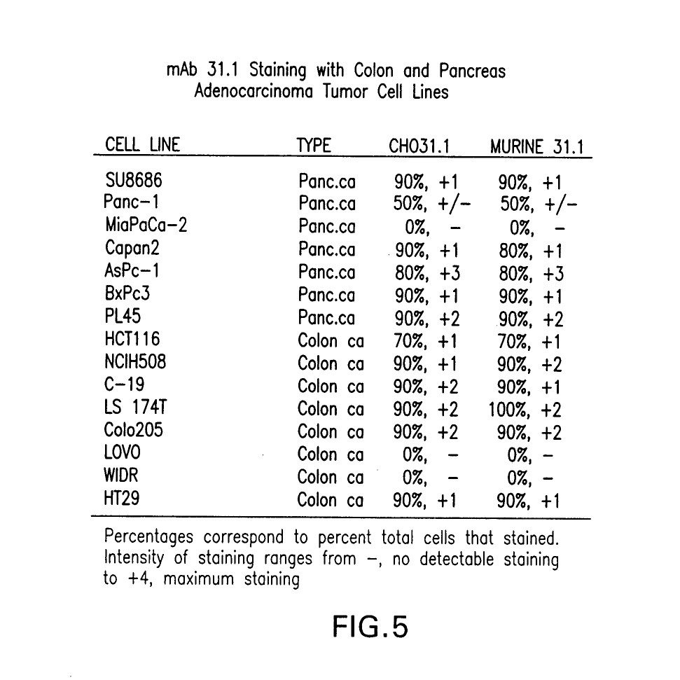

Immunohistochemistry of colon and pancreas adenocarcinoma tumor cell lines. Immunohistochemistry of various colon and pancreatic adenocarcinoma tumor cell lines purchased from ATCC was done to determine 1) the number and variety of cell lines that CHO 31.1 binds to and 2) the degree of binding of CHO 31.1 to specific cell lines. The results are shown in FIG. 5. CHO 31.1 binds to all but three of the cell lines examined. The percentage of cells that bind antibody as well as the intensity of staining (0, no staining, +3, strong staining) are shown. Murine 31.1 is the original pre-chimeric clone of mAb 31.1 and is shown for comparison. The data show that both the murine and chimeric forms of mAb 31.1 bind these cell lines in the same fashion.

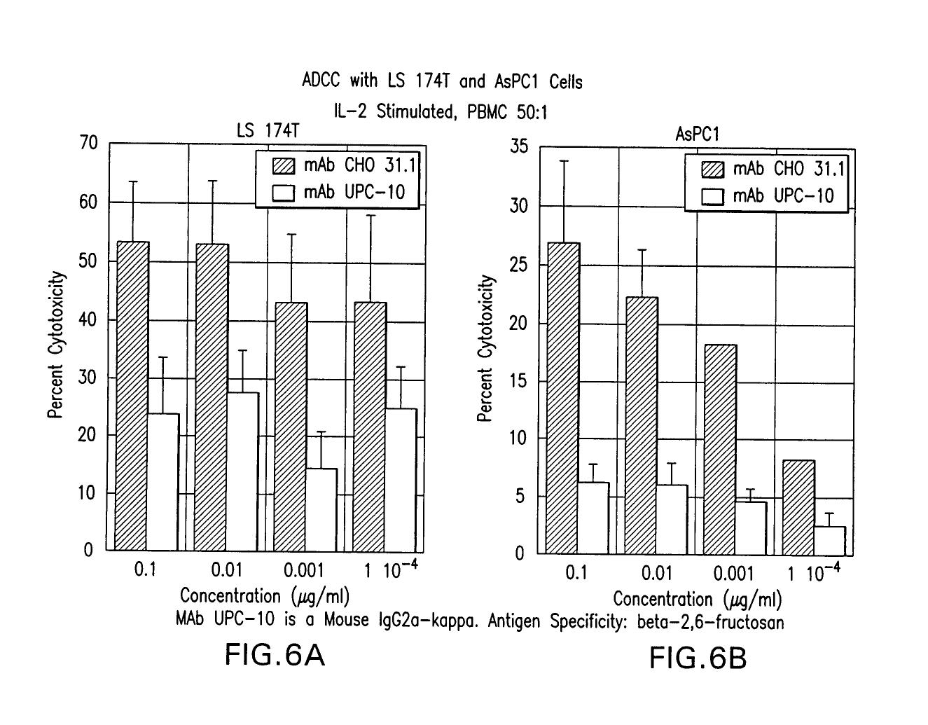

ADCC studies. Antibody dependent cellular toxicity (ADCC) is an in vitro functional assay. In this assay target cells are seeded in 96-well plates and incubated with either CHO 31.1 or a non-specific IgG1 antibody (UPC-10 used as control) and allowed to bind. Human effector cells are then added and incubated at 37° C. to allow for cytotoxicity events to occur. An enzymatic assay is then performed to determine the percentage of living cells remaining. FIG. 6A-B shows the results of ADCC studies using either colon carcinoma cell line LS 174T cells (FIG. 6A) or pancreatic carcinoma cell line AsPC1 (FIG. 6B) as targets. For colon carcinoma cells, there was an approximately 2 fold increase in cell cytotoxicity with CHO 31.1 MAb relative to control values. Against AsPC-1 cells, there was a greater than 5-fold increase in cell cytotoxicity with CHO 31.1 over control.

TABLE 1
Immunohistochemistry of Normal Tissues and Organs

| Tissue | # Samples | # Positive | Intensity¹ | Structure |
|---|---|---|---|---|
| Fallopian Tube | 3 | 0 | | |
| Ovary | 3 | 0 | | |
| Thyroid | 4 | 1 | +1 | Duct |
| Testicle | 2 | 0 | | |
| Brain | 3 | 0 | | |
| Jejunum | 3 | 1 | +3 | Mucosa |
| Muscle | 2 | 0 | | |
| Spleen | 3 | 0 | | |
| Appendix | 3 | 0 | | |
| Vaginal Mucosa | 3 | 0 | | |
| Esophagus | 3 | 0 | | |
| Lymph Node | 4 | 0 | | |
| Stomach | 3 | 2 | +3 | Mucosa |
| Liver | 4 | 1 | +/− | Epithelial |
| Salivary Gland | 3 | 3 | +2 | Duct & |
| Epithelial | | | | |
| Parotid | 2 | 1 | +2 | Duct |
| Prostate | 4 | 0 | | |
| Colon | 4 | 2 | +/− | Mucosa |
| Lung | 3 | 0 | | |
| Pancreas | 4 | 0 | | |
| Skin | 4 | 2 | +1 | Basement |
| Membrane | | | | |
| Heart | 7 | 0 | | |
| Breast | 5 | 0 | | |
| Bone Marrow | 3 | 0 | | |
| Adrenal | 3 | 1 | +/− | Epithelial |
| Bladder | 3 | 0 | | |
| Gall Bladder | 3 | 0 | | |
| Spinal Cord | 2 | 0 | | |
| Thymus | 7 | 1 | +/− | Epithelial |
| Endometrium | 1 | 0 | | |
| Tonsil | 3 | 0 | | |
| Placenta | 2 | 0 | | |
| White Blood Cells | 4 | 0 | | |
| Eye | 2 | 0 | | |
| Oral Mucosa | 1 | 0 | | |
| Kidney | 3 | 0 | | |
| Ileum | 2 | 0 | | |

¹Increase in positive immunohistochemical staining intensity is represented by increasing number values ranging from slightly above background (+/−) to heavily stained (+3).

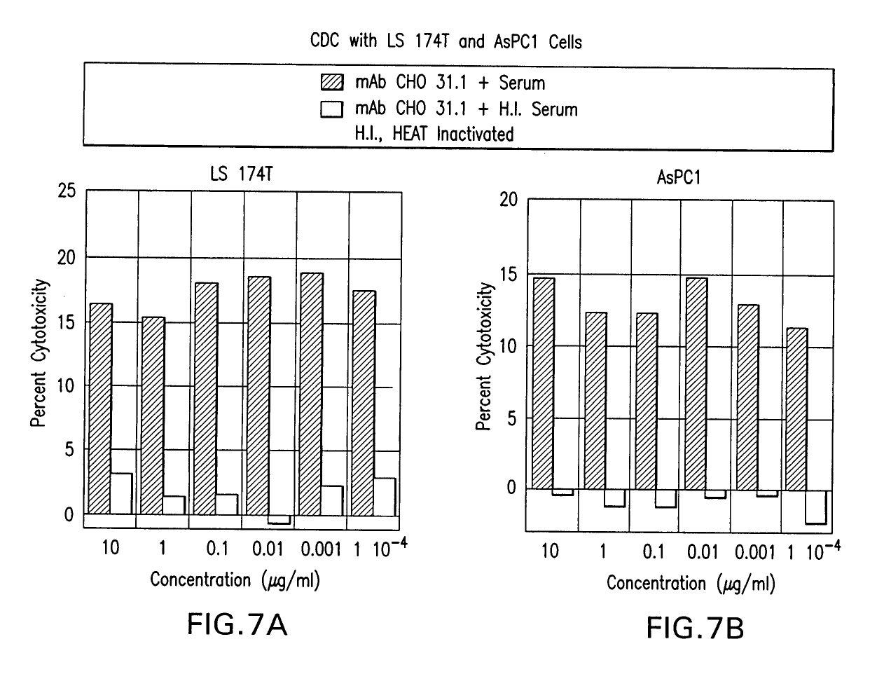

Compliment Dependent Cytotoxicity Studies. mAb CHO31.1 mediates tumor cell death through a CDC pathway when compared to heat inactivated serum controls. (Note: heat inactivation destroys compliment). FIG. 7A-B demonstrates that CHO 31.1 MAb was cytotoxic to colon carcinoma cell line LS 147T cells (FIG. 7A) as well as pancreatic carcinoma cell line AsPC1 cells (FIG. 7B).

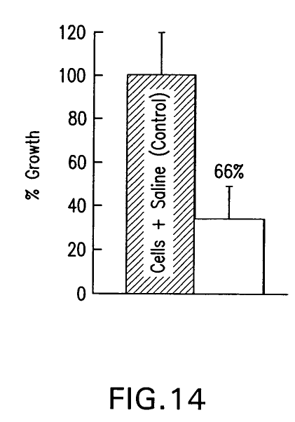

CHO 31.1 MAb inhibits colony formation in soft agar. In this assay ASPC-1 cells were first seeded in 12-well plates in triplicate at low cell density (250 cells/well). CHO 31.1 ( 10 ug/ml) was added to the media 24 hrs. after seeding. dPBS (same volume as experimental group) was used as a control. Colonies greater than 50 cells were counted 2 weeks later. Results (FIG. 14) show a significant reduction in colony formation in those wells in which mAb CHO 31.1 was added compared to controls.

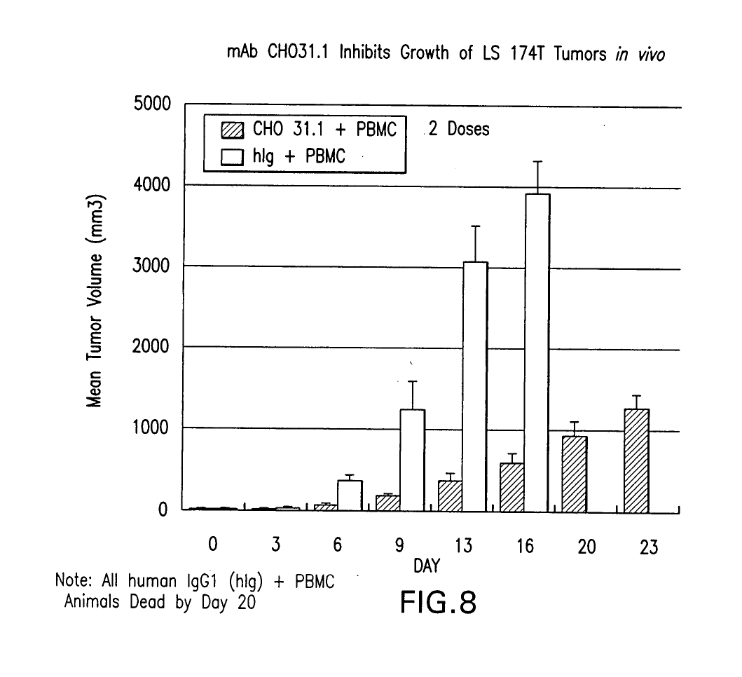

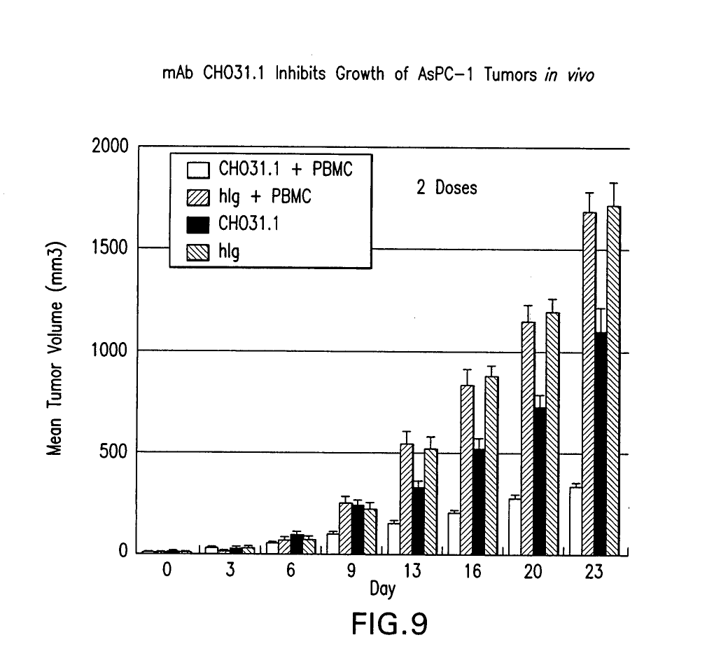

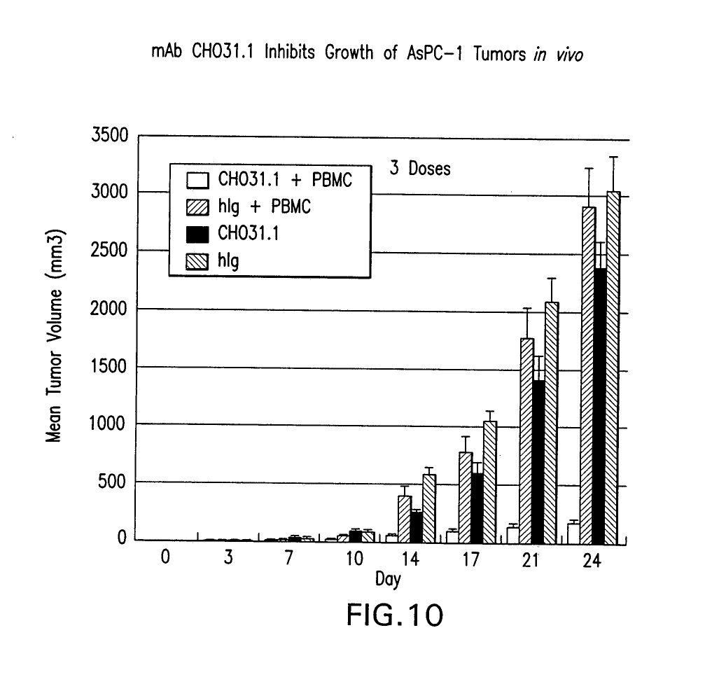

CHO 31.1 MAb inhibits tumor growth in vivo. CHO 31.1 inhibits the growth of both LS 174-T (FIG. 8) and ASPC-1 tumor cells (FIGS. 9 and 10) in mice. FIG. 8 demonstrates the ability of two doses of CHO 31.1 MAb to decrease growth of LS 1 74T tumors, where even after 23 days, the tumor burden in treated animals is only at a level observed in day 9 controls. FIGS. 9 and 10 show the effects of 2 and 3 doses, respectively, of CHO 31.1 MAb on growth of pancreatic AsPC-1 tumors. Mean tumor volume was significantly smaller in those animals which received CHO 31.1 combined with human effector cells (PBMC) as shown in red, followed by animals which received CHO 31.1 alone as shown in green, followed by animals which received either nonspecific human IgG1 alone or with PBMC as shown in black and blue, respectively. The effect of increasing dose in suppressing tumor growth is demonstrated by comparing FIGS. 9 and 10, where the graph is essentially ‘shifted to the right’ by about four days.

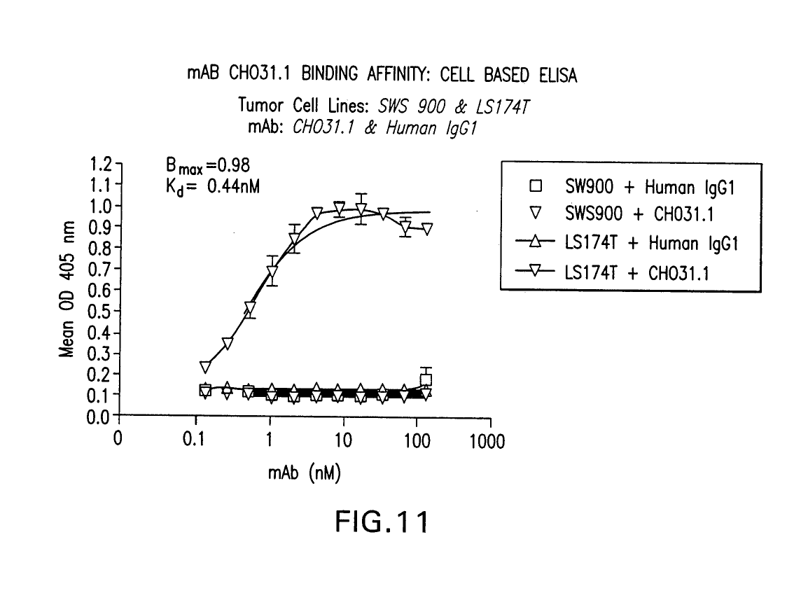

Cell based ELISA studies. A cell based ELISA was developed as a functional assay for CHO 31.1. In this assay target cells (e.g. LS 174-T, ASPC-1, and Capan-2) are seeded onto a 96-well plate, allowed to adhere, and then incubated with CHO 31.1. An alkaline phophatase conjugated goat-antihuman secondary antibody is used to develop the ELISA. SW900 (e.g. breast adenocarcinoma tumor cells) are used as a negative control cell line. This in vitro assay is useful in that it detects structural integrity and functionality of the variable region (required to bind target cell antigen) as well as the heavy chain constant regions (required for the binding of the goat-antihuman secondary antibody. This assay has also been used to demonstrate batch-to-batch consistency in research grade manufacturing. Results are shown in FIG. 11.

### EXAMPLE 3

### CHO 31.1 MAb and A33 MAb Both Bind to Cells Expressing A33 Antigen

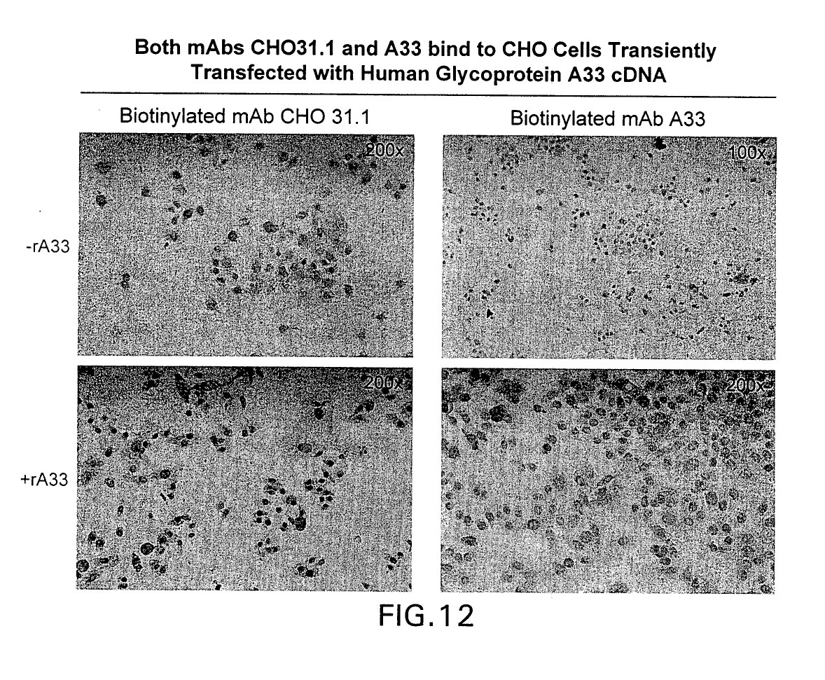

The human glycoprotein A33 cDNA was cloned from COLO205 cells by PCR and transiently expressed in CHO cells. Cells transiently expressing A33 antigen were then subjected to immunohistochemical analysis with mAbs CHO31.1 and A33. As shown in FIG. 12, both mAbs bind to CHO cells expressing A33 antigen (as seen in the +rA33 row) when compared to non-transfected cells (as seen in the −rA33 row) suggesting that both mAbs share a common antigen.

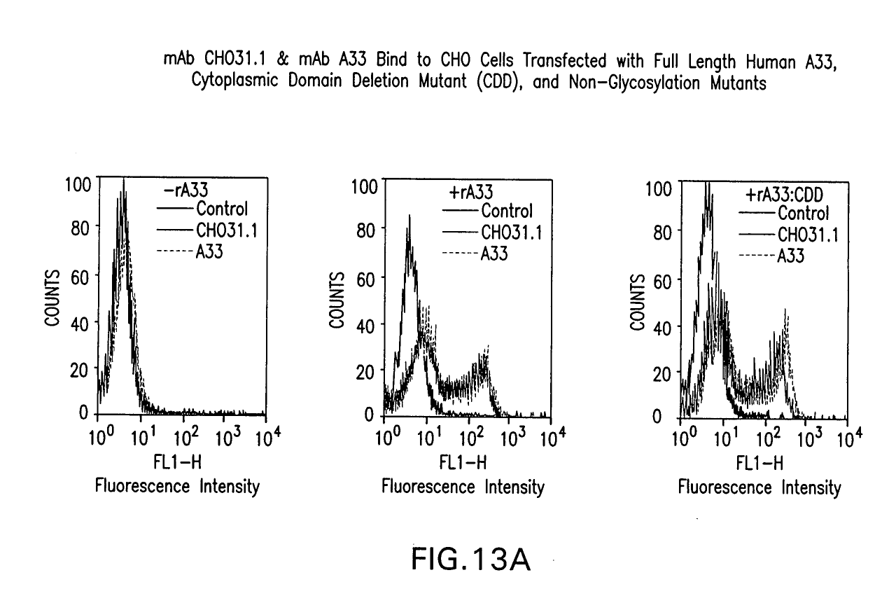

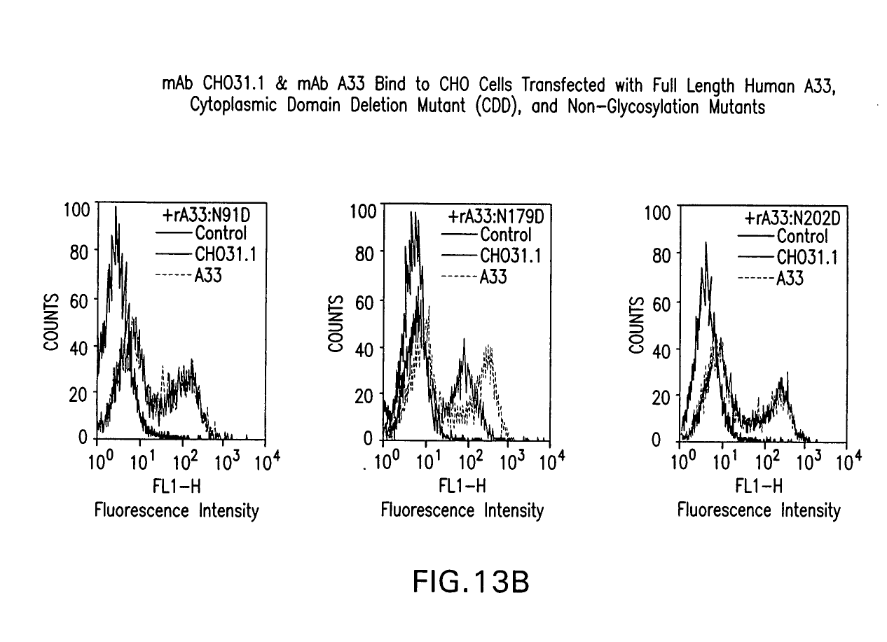

The panel of flow cytometry data shown in FIG. 13 corroborates the immunohistochemistry discussed above, in that both mAbs CHO31.1 and A33 bind to CHO cells transiently expressing the. A3 antigen. The panels show results of binding of the mAbs to full-length, truncated, and point mutated forms of the A33 antigen. Both mAbs bind in an identical fashion to both the full-length and mutated forms of the A33 antigen except for the N179D mutant as seen in the panel labeled +rA33:N179D. Here a distinct shift in the fluorescence intensity between the mAb CHO31.1 and mAb A33 spectra. is seen. These results suggest that mAbs CHO31.1 and A33 do not share the same epitope on the A33 antigen.

Various publications are cited herein, the contents of which are hereby incorporated by reference in their entireties.

What is claimed is:

1. A chinese hamster ovary cell expressing a 31.1 monoclonal antibody.

2. The chinese hamster ovary cell of claim 1, wherein the 31.1 monoclonal antibody has an sequence which is a variant of the amino acid sequence of the chimeric 31.1 monoclonal antibody as produced by cells deposited with the American Type Culture Collection and assigned accession number CRL-12316.

3. The chinese hamster ovary cell of claim 2, as deposited with the American Type Culture Collection and assigned Accession No. PTA-5712, as deposited on Dec. 30, 2003.

4. A composition comprising a 31.1 monoclonal antibody produced in the chinese hamster ovary cell of claim 1.

5. A composition comprising a 31.1 monoclonal antibody produced in the chinese hamster ovary cell of claim 2.

6. A composition comprising a 31.1 monoclonal antibody produced in the chinese hamster ovary cell of claim 3.

7. The composition of claim 4, which is free of serum-derived contaminants.

8. The composition of claim 5, which is free of serum-derived contaminants.

9. The composition of claim 6, which is free of serum-derived contaminants.

10. The composition of any of claims 4-9, wherein the monoclonal antibody is covalently or non-covalently linked to a reporter molecule.

11. The composition of any of claims 4-9, wherein the monoclonal antibody is covalently or non-covalently linked to a cytotoxic agent.

12. Use of a humanized 31.1 monoclonal antibody for the preparation of a pharmaceutical composition for the treatment of pancreatic cancer.

13. A method of treating pancreatic cancer, comprising administering, to a subject in need of such treatment, an effective amount of a humanized 31.1 monoclonal antibody.

14. A method for preparing 31.1 monoclonal antibody comprising:
(a) culturing chinese hamster ovary cells wherein said cells are genetically engineered to express said 31.1 monoclonal antibody; and
(b) collecting the 31.1 monoclonal antibody from the culture media


SEQUENCE LISTING (5 sequences)

SEQ ID NO: 1 - DNA, 10103 bp, Artificial Sequence
  artificial plasmid
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgatccactagtaacggccgccagtgtgctggaattcgccgccaccatggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacacggctacatatttctgtgcaagagcctactatggtaaatactttgactactggggccaaggcaccactctcacagtctcttcagcctccaccaagggcccatcggtcttccccttggcaccctcgagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatttcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggttctgcacaaccactacacgcagaagagcctctccctgtctccgggtaaatgagcggccgctcgactattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcacctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagactaacactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtacgtttcagctccagcttggtcccagcaccgaacgtgagcggagagctataatcctgctgacagaaataaactgccaggtcttcagcctgcacagtgctgatggtgaaagtgaaatccgtcccatatccactgccagtgaagcgatcagggactccagtgtagcgattggatgcatagtatatcagcagtttaggagactgccctggtttctgttggtaccaagctacatcattactcacactctgactggccttgcaggttatggtaaccctgtctcctgctgatacaagcaggaatttgggagtctgggtcatcacaatactcccatgagcaccagacacacagagcagtagaaatacgaagacctgggtctgtgacttcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggctagagcattggggggggggacagctcagggctgcgatttcgcgccaaacttgacggcaatcctagcgtgaaggctggtaggattttatccccgctgccatcatggttcgaccattgaactgcatcgtcgccgtgtcccaagatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 2 - DNA, 1413 bp, Artificial Sequence
  Synthetic plasmid
gccgccaccatggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacacggctacatatttctgtgcaagagcctactatggtaaatactttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggaccctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccatgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 467 aa, Artificial Sequence
  Synthetic peptide
MAWVWTLLFLMAAAQSAQAQIQLVQSGPELKKPGETVKISCKASGYTFTNYGMNWVKQAPGKGLKWMGWINTYTGEPTYADDFKGRFAFSLETSASTAYLQINNLKNEDTATYFCARAYYGKYFDYWGQGTTLTVSSASTKGPSVFPLAPSSKSTSGGTAALGCLVKDYFPEPVTVSWNSGALTSGVHTFPAVLQSSGPYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTMPPVLDSDGSFFLYSKLTVDKSRWQQGNVFSCSVMHEALHNHYTQKSLSLSPGK

SEQ ID NO: 4 - DNA, 714 bp, Artificial Sequence
  Synthetic plasmid
gccgccaccatgaagtcacagacccaggtcttcgtatttctactgctctgtgtgtctggtgctcatgggagtattgtgatgacccagactcccaaattcctgcttgtatcagcaggagacagggttaccataacctgcaaggccagtcagagtgtgagtaatgatgtagcttggtaccaacagaaaccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctccgctcacgttcggtgctgggaccaagctggagctgaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 234 aa, Artificial Sequence
  Synthetic peptide
MKSQTQVFVFLLLCVSGAHGSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC